(19) United States
(12) Patent Application Publication
(10) Pub. No.: US 2026/0157677 A1
(43) Pub. Date: Jun. 11, 2026

(54) THREE DIMENSIONAL TOOL FOR ECG ST SEGMENT MEASUREMENTS, REPRESENTATION, AND ANALYSIS

(71) Applicant: Drägerwerk AG & Co. KGaA, Lübeck (DE)

(72) Inventors: Rafael Andree Zuaznabar, Andover, MA (US); Rajesh S. Rane, Andover, MA (US)

(73) Assignee: Drägerwerk AG & Co. KGaA, Lübeck (DE)

(21) Appl. No.: 18/706,591

(22) PCT Filed: Oct. 27, 2022
(86) PCT No.: PCT/IB2022/060345

§ 371 Date: May 1, 2024

Related U.S. Application Data
(60) Provisional application No. 63/274,592, filed on Nov. 2, 2021.

(51) Int. Cl. A61B 5/339; A61B 5/358; G06T 19/20

(52) U.S. Cl. CPC A61B 5/339; A61B 5/358; G06T 19/20; G06T 2200/24; G06T 2210/41; G06T 2219/2012; G06T 2219/2016

(57) ABSTRACT

A physiological monitoring device includes a sensor interface configured to receive sensor signals from a plurality of electrocardiogram (ECG) sensors connected to a patient; a display configured to display information related to the patient; and at least one processor configured to derive a plurality of ECG signals from the sensor signals. Each ECG signal is a cardiac cycle waveform associated with a different region of a heart. The at least one processor is configured to measure an ST segment of each ECG signal in real time to acquire a plurality of real-time ST segment measurements and control the displayed information based on the plurality of real-time ST segment measurements. The displayed information includes a rotatable 3D anatomical representation of the heart that includes a plurality of visually distinct regions associated with the plurality of ECG signals and change in appearance based on the plurality of real-time ST segment measurements.

### BACKGROUND

Patient monitors are devices that are configured to receive physiological data from another device and either display a patient's physiological data, monitor a patient's physiological data, or both. A patient monitor may be configured to be worn by a patient, may be a hand-held device, may be docked to or undocked from a larger unit such as a monitor mount, and, thus, may be transportable. For example, a monitor mount may be a larger patient monitor or a console that has a docking interface or docking receptacle to which the patient monitor can be removably docked.

A patient monitor may be implemented to monitor cardiac signals from a patient via electrocardiogram (ECG) sensors connected to an ECG lead set. Commonly used ECG lead set configurations include 3-lead, 5-lead, 6-lead, 7-lead, 8-lead, 12-lead, 15-lead, and 16-lead configurations. In a 12-lead ECG configuration, for example, ten electrodes (i.e., sensors) are placed on predetermined locations of the skin of the patient body and extrapolated into twelve lead measurements. The overall magnitude of the heart's electrical potential is then measured from twelve different angles (“leads”). In this way, the overall magnitude and direction of the heart's electrical activity is captured throughout the heartbeat. In a 5-lead ECG configuration, five electrodes are used and extrapolated into five lead measurements. In a 14-lead configuration, twelve electrodes are used and extrapolated into sixteen lead measurements. In a 16-lead configuration, fourteen electrodes are used and extrapolated into sixteen lead measurements.

The ST segment on an ECG normally represents an electrically neutral, isoelectric section of the ECG complex between ventricular depolarization (QRS complex) and repolarization (T wave). In other words, it corresponds to the area from the end of the QRS complex to the beginning of the T wave of the ECG waveform. In clinical terms, the ST segment represents the period in which the myocardium maintains contraction to expel blood from the ventricles. However, the ST segment can take on various waveform morphologies that may indicate benign or clinically significant injury or abuse to the myocardium. Understanding the differential diagnosis for variations in the ST segment is critical for clinical management as it can influence treatment. For example, a normal ST segment is usually isoelectric (i.e., flat on the baseline, neither positive nor negative), but it may be slightly elevated or depressed normally (usually by less than 1 mm). ST segments more than 1 to 2 mm above baseline are termed elevated, and those more than 1 to 2 mm lower than baseline are termed depressed. A depressed ST segment may indicate coronary ischemia. An elevated ST segment may indicate acute myocardial infarction.

Current patient monitors are capable of performing ST measurements to detect elevations or depressions present at the ST segment and output the measurements either in the form of text (e.g., numerical values) or as a waveform. Some patient monitors provide a two-dimensional (2D) graphical display that focus on localized measured values shown on a clock-like display for the limb and augmented leads and a second clock-like display for the precordial leads. It is delegated to the user to match those two clocks to have all the necessary information making this tool difficult to read and interpret and to use on a continuous manner. The difficulty in interpreting this type of representation of the ST segment measurements often required specialized personnel on site, who may not always be available.

Additionally, current patient monitors do not provide a complete view of various regions of the heart that are being affected by an abnormal ST segment or a progression of changes within those regions over time. Thus, current ST segment monitoring systems could lead to inefficient treatment. Having a three-dimensional (3D) clinical monitor that can show the current status of different regions of the heart that are being monitored in real-time on a 3D anatomical representation of the heart (e.g., a 3D image) may be desirable. It may also be desirable to provide a graphical representation that is easier to read and interpret such that abnormalities of the heart during an ST segment measurement are highlighted and easily observable and understandable to enable a focused treatment.

### SUMMARY

One or more embodiments provide a physiological monitoring device includes a sensor interface configured to receive sensor signals from a plurality of electrocardiogram (ECG) sensors connected to a patient; a display configured to display information related to the patient; and at least one processor configured to derive a plurality of ECG signals from the sensor signals. Each ECG signal is a cardiac cycle waveform associated with a different region of a heart. The at least one processor is configured to measure an ST segment of each ECG signal in real time to acquire a plurality of real-time ST segment measurements and control the displayed information based on the plurality of real-time ST segment measurements. The displayed information includes a rotatable 3D anatomical representation of the heart that includes a plurality of visually distinct regions associated with the plurality of ECG signals and change in appearance based on the plurality of real-time ST segment measurements.

One or more embodiments provide a physiological monitoring device, including: a sensor interface configured to receive sensor signals from a plurality of ECG sensors connected to a patient; a display configured to display information related to the patient; and at least one processor configured to receive the sensor signals and derive a plurality of ECG signals therefrom, wherein each ECG signal is a cardiac cycle waveform associated with a different region of a heart, wherein the at least one processor is further configured to measure an ST segment of each ECG signal in real time to acquire a plurality of real-time ST segment measurements and control the displayed information based on the plurality of real-time ST segment measurements. The displayed information includes a rotatable 3D anatomical representation of the heart that includes a plurality of visually distinct regions that are associated with the plurality of ECG signals, wherein each visually distinct region is associated with a different ECG signal of the plurality of ECG signals. Each visually distinct region includes at least one visual characteristic that is configured to change based on a real-time ST segment measurement of the ECG signal associated therewith. Each measured ST segment of each ECG signal is identifiable as a depressed ST segment, a normal ST segment, or an elevated ST segment according to the at least one visual characteristic of a corresponding visually distinct region of the plurality of visually distinct regions. The at least one processor is configured to adapt the at least one visual characteristic of each visually distinct region in real-time based on the real-time ST segment measurement associated therewith to visually indicate that the real-time ST segment measurement associated therewith is depressed, normal, or elevated.

One or more embodiments provide an ECG system, including: a sensor interface configured to receive sensor signals from a plurality of ECG sensors connected to a patient; a display configured to display information related to the patient; and at least one processor configured to receive the sensor signals and derive a plurality of ECG signals therefrom, wherein each ECG signal is a cardiac cycle waveform associated with a different region of a heart, wherein the at least one processor is further configured to measure an ST segment of each ECG signal in real time to acquire a plurality of real-time ST segment measurements and control the displayed information based on the plurality of real-time ST segment measurements. The displayed information includes a rotatable 3D anatomical representation of the heart that includes a plurality of visually distinct regions that are associated with the plurality of ECG signals, wherein each visually distinct region is associated with a different ECG signal of the plurality of ECG signals. Each visually distinct region includes at least one visual characteristic that is configured to change based on a real-time ST segment measurement of the ECG signal associated therewith. Each measured ST segment of each ECG signal is identifiable as a depressed ST segment, a normal ST segment, or an elevated ST segment according to the at least one visual characteristic of a corresponding visually distinct region of the plurality of visually distinct regions. The at least one processor is configured to adapt the at least one visual characteristic of each visually distinct region in real-time based on the real-time ST segment measurement associated therewith to visually indicate that the real-time ST segment measurement associated therewith is depressed, normal, or elevated.

One or more embodiments provide a method of monitoring ST segments in a plurality of plurality of ECG signals. The method includes: receiving sensor signals from a plurality of ECG sensors connected to a patient; displaying information related to the patient; deriving a plurality of ECG signals from the sensor signals, wherein each ECG signal is a cardiac cycle waveform associated with a different region of a heart; measuring an ST segment of each ECG signal in real time to acquire a plurality of real-time ST segment measurements; controlling the displayed information based on the plurality of real-time ST segment measurements, wherein the displayed information includes a rotatable 3D anatomical representation of the heart that includes a plurality of visually distinct regions that are associated with the plurality of ECG signals, wherein each visually distinct region is associated with a different ECG signal of the plurality of ECG signals, wherein each visually distinct region includes at least one visual characteristic that is configured to change based on a real-time ST segment measurement of the ECG signal associated therewith, wherein each measured ST segment of each ECG signal is identifiable as a depressed ST segment, a normal ST segment, or an elevated ST segment according to the at least one visual characteristic of a corresponding visually distinct region of the plurality of visually distinct regions, and wherein controlling the displayed information includes adapting the at least one visual characteristic of each visually distinct region in real-time based on the real-time ST segment measurement associated therewith to visually indicate that the real-time ST segment measurement associated therewith is depressed, normal, or elevated.

### BRIEF DESCRIPTION OF THE DRAWINGS

In the drawings, like reference numbers generally indicate identical, functionally similar, and/or structurally similar elements.

FIG. 1 shows a physiological monitoring system according to one or more embodiments;

FIG. 2A is a plot of an example cardiac cycle waveform of an ECG signal showing the Q, R, S, and T waves, the J-point, and the ST segment according to one or more embodiments;

FIG. 2B is a plot of an example cardiac cycle waveform of an ECG signal showing a ST elevation according to one or more embodiments;

FIGS. 3A and 3B illustrate a display of a patient monitor configured to display a rotatable 3D anatomical representation of a heart that includes visually identifiable regions each corresponding to a different ECG lead measurement according to one or more embodiments;

FIG. 3C is a frontal view of the 3D anatomical representation of a heart adapted to an 8-lead ECG configuration with heart regions AVF, AVL, AVR, II, III, V2 and V4 being visible on a display according to one or more embodiments; and

FIG. 3D illustrates a display of a patient monitor with a pattern of change mode enabled according to one or more embodiments.

### DETAILED DESCRIPTION

In the following, details are set forth to provide a more thorough explanation of the embodiments. However, it will be apparent to those skilled in the art that embodiments may be practiced without these specific details. In other instances, well-known structures and devices are shown in block diagram form or in a schematic view rather than in detail in order to avoid obscuring the embodiments. In addition, features of the different embodiments described hereinafter may be combined with each other, unless specifically noted otherwise. For example, variations or modifications described with respect to one of the embodiments may also be applicable to other embodiments unless noted to the contrary.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

Directional terminology, such as “top”, “bottom”, “below”, “above”, “front”, “behind”, “back”, “leading”, “trailing”, etc., may be used with reference to the orientation of the figures being described. Because parts of embodiments can be positioned in a number of different orientations, the directional terminology is used for purposes of illustration. It is to be understood that other embodiments may be utilized, and structural or logical changes may be made without departing from the scope defined by the claims. The following detailed description, therefore, is not to be taken in a limiting sense. Directional terminology used in the claims may aid in defining one element's spatial or positional relation to another element or feature, without being limited to a specific orientation.

Instructions may be executed by one or more processors, such as one or more central processing units (CPU), digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein refers to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. A “controller,” including one or more processors, may use electrical signals and digital algorithms to perform its receptive, analytic, and control functions, which may further include corrective functions. Thus, a controller is a specific type of processing circuitry, comprising one or more processors and memory, that implements control functions by way of generating control signals.

A sensor refers to a component which converts a physical quantity to be measured to an electric signal, for example, a current signal or a voltage signal. The physical quantity may for example comprise electromagnetic radiation (e.g., photons of infrared or visible light), a magnetic field, an electric field, a pressure, a force, a temperature, a current, or a voltage, but is not limited thereto.

Signal conditioning, as used herein, refers to manipulating an analog signal in such a way that the signal meets the requirements of a next stage for further processing. Signal conditioning may include converting from analog to digital (e.g., via an analog-to-digital converter), amplification, filtering, converting, biasing, range matching, isolation and any other processes required to make a sensor output suitable for processing after conditioning.

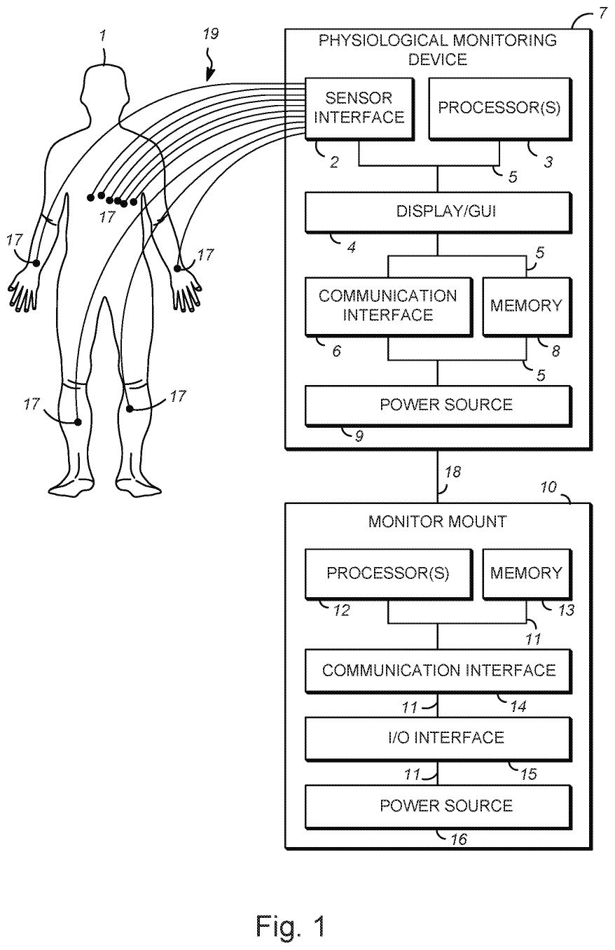

FIG. 1 shows a physiological monitoring system according to one or more embodiments. As shown in FIG. 1, the system includes a patient monitor 7 (i.e., a physiological monitoring device) capable of receiving physiological data from various sensors 17 connected to a patient 1. A patient monitor is a device that is configured to receive physiological data from another device and either display a patient's physiological data, monitor a patient's physiological data, or both. A patient monitor may be configured to be worn by a patient, may be a hand-held device, may be docked to or undocked from a larger unit such as a monitor mount, and, thus, may be transportable or non-transportable. For example, a monitor mount may be a larger patient monitor or a console that has a docking interface or docking receptacle to which the patient monitor can be removably docked.

A patient monitor may have memory and one or more processors that are co-operatively configured to receive sensor data from one or more sensors, process the sensor data in order to monitor one or more physiological parameters, including deriving one or more of the physiological parameters from the sensor data, and control a display to display the monitored physiological parameters by one or more graphical or textual representations. Additionally, or alternatively, memory and at least one processor may be located on a network (e.g., on an external server or computer) that provides additional processing power and capabilities that may not be present on the patient monitor. In this case, the patient monitor may transmit the sensor data on a network to an external processor, which in turn derives one or more of the physiological parameters from the sensor data, performs monitoring functions on the physiological parameters, and transmits monitored data and/or display data back to the patient monitor for display on a display of the patient monitor. The transmissions on the network may be through wireless communication channels, wired communication channels, or a combination thereof, but should be sufficiently fast to enable monitoring and display of data in real-time.

In general, it is contemplated by the present disclosure that the patient monitor 7 includes electronic components and/or electronic computing devices operable to receive, transmit, process, store, and/or manage patient data and information associated performing the functions of the system, which encompasses any suitable processing device adapted to perform computing tasks consistent with the execution of computer-readable instructions stored in a memory or a computer-readable recording medium.

Further, any, all, or some of the computing devices in the patient monitor 7 may be adapted to execute any operating system, including Linux, UNIX, Windows Server, etc., as well as virtual machines adapted to virtualize execution of a particular operating system, including customized and proprietary operating systems. The patient monitor 7 may be further equipped with components to facilitate communication with other computing devices over one or more network connections, which may include connections to local and wide area networks, wireless and wired networks, public and private networks, and any other communication network enabling communication in the system.

As shown in FIG. 1, the patient monitor 7 is, for example, a patient monitor implemented to monitor various physiological parameters of the patient 1 via the sensors 17. The patient monitor 7 includes a sensor interface 2, one or more processors 3, a display/graphical user interface (GUI) 4, a communication interface 6, a memory 8, and a power source 9. The sensor interface 2 can be implemented in hardware or combination of hardware and software and is used to connect via wired and/or wireless connections 19 to one or more sensors 17 for gathering physiological data from the patient 1. The sensors 17 may be physiological sensors and/or medical devices configured to measure one or more of the physiological parameters and output the measurements via a corresponding one or more connections 19 to the sensor interface 2. Thus, the connections 19 represent one or more wired or wireless communication channels configured to at least transmit sensor data from a corresponding sensor 17 to the sensor interface 2.

By way of example, sensors 17 may include electrodes that are attached to the patient 1 for reading electrical signals generated by or passed through the patient 1. Sensors 17 may be configured to measure vital signs, measure electrical stimulation, measure brain electrical activity such as in the case of an electroencephalogram (EEG), measure blood characteristics using absorption of light, for example, blood oxygen saturation fraction from absorption of light at different wavelengths as it passes through a finger, measure a carbon dioxide (CO₂) level and/or other gas levels in an exhalation stream using infrared spectroscopy, measure oxygen saturation on the surface of the brain or other regions, measure cardiac output from invasive and noninvasive blood pressure, measure temperature, measure induced electrical potentials over the cortex of the brain, measure blood oxygen saturation from an optical sensor coupled by fiber to the tip of a catheter. The data signals from the sensors 17 include, for example, sensor data related to an electrocardiogram (ECG), non-invasive peripheral oxygen saturation (SpO2), non-invasive blood pressure (NIBP), body temperature, end tidal carbon dioxide (etCO2), apnea detection, and/or other physiological data, including those described herein.

The one or more processors 3 are used for controlling the general operations of the patient monitor 7, as well as processing sensor data received by the sensor interface 2. Each one of the one or more processors 3 can be, but are not limited to, a central processing unit (CPU), a hardware microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for controlling the operation and performing the functions of the patient monitor 7.

The processing of sensor data may include performing signal conditioning on the sensor data, monitoring the sensor data, deriving physiological parameters from the sensor data, monitoring the physiological parameters, comparing the sensor data and/or the physiological parameters to one or more thresholds, generating alarms based on the monitoring/comparison results, generating visual representations of the sensor data and/or physiological parameters, and displaying the visual representations, but is not limited thereto.

The display/GUI 4 is configured to display various patient data, sensor data, physiological parameters, and hospital or patient care information, and includes a user interface implemented for allowing interaction and communication between a user and the patient monitor 7. The display/GUI 4 includes, but is not limited to a liquid crystal display (LCD), cathode ray tube (CRT) display, thin film transistor (TFT) display, light-emitting diode (LED) display, high definition (HD) display, or other similar display device that may or may not include touch screen capabilities. The display/GUI 4 also provides a means for inputting instructions or information directly to the patient monitor 7. The patient information displayed can, for example, relate to the measured physiological parameters of the patient 1 (e.g., blood pressure, heart related information, pulse oximetry, respiration information, etc.) as well as information related to the transporting of the patient 1 (e.g., transport indicators). The patient monitor 7 may also be connectable to additional user input devices, such as a keyboard or a mouse.

The communication interface 6 enables the patient monitor 7 to directly or indirectly communicate with one or more computing networks and devices, including one or more sensors 17, workstations, consoles, computers, monitoring equipment, alert systems, and/or mobile devices (e.g., a mobile phone, tablet, or other hand-held display device). The communication interface 6 can include various network cards, interfaces, communication channels, cloud, antennas, and/or circuitry to enable wired and wireless communications with such computing networks and devices. The communication interface 6 can be used to implement, for example, a Bluetooth connection, a cellular network connection, and/or a Wi-Fi connection with such computing networks and devices. Example wireless communication connections implemented using the communication interface 6 include wireless connections that operate in accordance with, but are not limited to, IEEE802.11 protocol, a Radio Frequency For Consumer Electronics (RF4CE) protocol, and/or IEEE802.15.4 protocol (e.g., ZigBee protocol).

Additionally, the communication interface 6 can enable direct (e.g., device-to-device) communications (e.g., messaging, signal exchange, etc.) to the patient monitor 7 using, for example, a universal serial bus (USB) connection or other communication protocol interface. The communication interface 6 can also enable direct device-to-device connection to other device such as to a tablet, computer, or similar electronic device; or to an external storage device or memory.

The memory 8 can be a single memory device or one or more memory devices at one or more memory locations that include, but is not limited to, a random access memory (RAM), a memory buffer, a hard drive, a database, an erasable programmable read only memory (EPROM), an electrically erasable programmable read only memory (EEPROM), a read only memory (ROM), a flash memory, hard disk, various layers of memory hierarchy, or any other non-transitory computer readable medium. The memory 8 can be used to store any type of instructions and patient data associated with algorithms, processes, or operations for controlling the general functions and operations of the patient monitor 7.

The power source 9 can include a self-contained power source such as a battery pack and/or include an interface to be powered either directly or indirectly through an electrical outlet. The power source 9 can also be a rechargeable battery that can be detached allowing for replacement. In the case of a rechargeable battery, a small built-in back-up battery (or super capacitor) can be provided for continuous power to be provided to the patient monitor 7 during battery replacement. Communication between the components of the patient monitor 7 (e.g., components 2, 3, 4, 6, 8, and 9) are established using an internal bus 5.

Accordingly, the patient monitor 7 is attached to one or more of several different types of sensors 17 configured to measure and readout physiological data related to the patient 1 (e.g., as shown on the left side of FIG. 1). One or more sensors 17 may be attached to patient monitor 7 by, for example, a wired connection coupled to the sensor interface 2. Additionally, or alternatively, one or more sensors 17 may be a wireless sensor that is communicatively coupled to the patient monitor 7 via the communication interface 6, which includes circuity for receiving data from and sending data to one or more devices using, for example, a Wi-Fi connection, a cellular network connection, and/or a Bluetooth connection.

As shown in FIG. 1, the patient monitor 7 may be connected to a monitor mount 10 via a connection 18 that establishes a communication connection between, for example, the respective communications interfaces 6, 14 of the devices 7, 10. The connection 18 is an interface that enables the monitor mount 10 to detachably secure the patient monitor 7 to the monitor mount 10. In this regard, “detachably secure” means that the monitor mount 10 can receive and secure the patient monitor 7, but the patient monitor 7 can also be removed or undocked from the monitor mount 10 by a user when desired. In other words, the patient monitor 7 can be removably docked or removably mounted to the monitor mount 10 and the connection 18 forms an electrical connection between the devices 7, 10 for enabling communication therebetween.

The connection 18 may enable the patient monitor 7 to transmit sensor data acquired from sensors 17 to the monitor mount 10 to be processed and analyzed by processor(s) 12 integrated within the monitor mount 10. The connection 18 may enable the monitor mount 10 to transmit processed sensor data, measured physiological parameters derived from the sensor data, and/or control signals to the patient monitor 7. Control signals may include instructions for controlling the display of data, images, graphics, text, indicators, and/or icons on the display 4 of the patient monitor 7. These control signals may be received by a processor 3 of the patient monitor 7, which in turn controls the imagery on the display 4 accordingly. The monitor mount 10 may also be connected to a network (e.g., a hospital network) and cooperatively exchanges information therewith for the processing of data and the generation of control signals.

The connection 18 may also enable the transmission of power from the monitor mount 10 to the patient monitor 7 for charging the power source 9. The connection 18 may also enable the patient monitor 7 to detect whether it is in a docked or undocked state. Thus, the connection 18 may further enable the patient monitor 7 via the interface to detect an undocking event and a docking event by detecting an electrical connection or an optical link, or an absence thereof, between the patient monitor 7 and the monitor mount 10.

The connection 18 may include, but is not limited to, a USB connection, a parallel connection, a serial connection, a coaxial connection, a High-Definition Multimedia Interface (HDMI) connection, an optical connection, and/or any other electrical connection configured to connect electronic devices and transmit data and/or power therebetween.

The monitor mount 10 includes one or more processors 12, a memory 13, a communications interface 14, an I/O interface 15, and a power source 16. The one or more processors 12 are used for controlling the general operations of the monitor mount 10 and may be further used to controller one or more operations of the patient monitor 7 when mounted to the monitor mount 10. Each one of the one or more processors 12 can be, but are not limited to, a CPU, a hardware microprocessor, a multi-core processor, a single core processor, an FPGA, a microcontroller, an ASIC, a DSP, or other similar processing device capable of executing any type of instructions, algorithms, or software for controlling the operation and performing the functions of the monitor mount 10.

The memory 13 can be a single memory or one or more memories or memory locations that include, but are not limited to a RAM, a memory buffer, a hard drive, a database, an EPROM, an EEPROM, a ROM, a flash memory, hard disk, or various layers of memory hierarchy, or any other non-transitory computer readable medium. The memory 13 can be used to store any type of instructions associated with algorithms, processes, or operations for controlling the general functions and operations of the monitor mount 10.

The communications interface 14 allows the monitor mount 10 to communicate with one or more computing networks and devices (e.g., the patient monitor 7, workstations, consoles, computers, monitoring equipment, alert systems, and/or mobile devices (e.g., a mobile phone, tablet, or other hand-held display device). The communications interface 14 can include various network cards, interfaces, communication channels, antennas, and/or circuitry to enable wired and wireless communications with such computing networks and devices. The communications interface 14 can also be used to implement, for example, a Bluetooth connection, a cellular network connection, cloud-based connection, and a Wi-Fi connection. Example wireless communication connections implemented using the communications interface 14 include wireless connections that operate in accordance with, but are not limited to, IEEE802.11 protocol, a Radio Frequency For Consumer Electronics (RF4CE) protocol, and/or IEEE802.15.4 protocol (e.g., ZigBee protocol). In essence, any wireless communication protocol may be used.

The communications interface 14 can also enable direct (i.e., device-to-device) communications (e.g., messaging, signal exchange, etc.) such as from the monitor mount 10 to the patient monitor 7 using, for example, the connection 18. The communications interface 14 can enable direct (i.e., device-to-device) to other device such as to a tablet, PC, or similar electronic device; or to an external storage device or memory.

The I/O interface 15 can be an interface for enabling the transfer of information between monitor mount 10, one or more physiological monitoring devices 7, and external devices such as peripherals connected to the monitor mount 10 that need special communication links for interfacing with the one or more processors 12. The I/O interface 15 can be implemented to accommodate various connections to the monitor mount 10 that include, but is not limited to, a USB connection, a parallel connection, a serial connection, a coaxial connection, an HDMI connection, or any other electrical connection configured to connect electronic devices and transmit data therebetween.

The power source 16 can include a self-contained power source such as a battery pack and/or include an interface to be powered through an electrical outlet (either directly or by way of the patient monitor 7). The power source 16 can also be a rechargeable battery that can be detached allowing for replacement. Communication between the components of the monitor mount 10 (e.g., components 12, 13, 14, 15 and 16) are established using an internal bus 11.

Embodiments described herein are directed to ECG data and the display of ECG data on the display/GUI 4. Thus, the data signals from the sensors 17 of the described embodiments are related to an ECG. The data signals received from an ECG sensor (e.g., an electrode) can be analog signals. For example, the data signals for the ECG are input to the sensor interface 2, which can include an ECG data acquisition circuit. Both the ECG data acquisition circuit may include amplifying and filtering circuity as well as analog-to-digital (A/D) circuity that converts the analog signal to a digital signal using amplification, filtering, and A/D conversion methods. In the event that an ECG sensor is a wireless sensor, the sensor interface 2 may receive the data signals from a wireless communication module. Thus, a sensor interface is a component configured to interface with one or more sensors 17 and receive sensor data therefrom.

The processing performed by the ECG data acquisition circuit may generate analog data waveforms or digital data waveforms that are analyzed by at least one processor of processors 3. For example, a processor 3 may analyze digital waveforms derived from sensor data to identify certain digital waveform characteristics and threshold levels indicative of conditions (abnormal and normal) of the patient 1 using one or more monitoring methods. A monitoring method may include comparing an analog or a digital waveform characteristic or an analog or digital value to one or more threshold values and generating a comparison result based thereon. The processor 3 is, for example, an FPGA, an ASIC, a DSP, a microcontroller, or similar processing device. The processor 3 includes a memory or uses a separate memory 8. The memory is, for example, a RAM, a memory buffer, a hard drive, a database, an EPROM, an EEPROM, a ROM, a flash memory, a hard disk, or any other non-transitory computer readable medium. The processor 3 may be further configured to control the display 4 to display certain images, graphics, text, and other indictors and icons by, for example, providing display data and/or control data thereto.

The memory stores software or algorithms with executable instructions and the processor 3 can execute a set of instructions of the software or algorithms in association with executing different operations and functions of the patient monitor 7 such as analyzing the digital data waveforms related to the data signals received from the sensors 17.

Specifically, the patient monitor 7 is configured to be connected to an ECG lead set and generate a number of lead measurements based on the number of sensors 17 attached to the body of the patient 1. In a 12-lead ECG configuration, for example, ten electrodes (i.e., sensors 17) are placed on predetermined locations of the skin of the patient body and extrapolated into twelve lead measurements. The overall magnitude of the heart's electrical potential is then measured from twelve different angles (“leads”) and is recorded over a period of time (e.g., 10 seconds). In this way, the overall magnitude and direction of the heart's electrical activity is captured throughout the heartbeat. In a 5-lead ECG configuration, five electrodes are used and extrapolated into five lead measurements. In a 16-lead configuration, fourteen electrodes are used and extrapolated into sixteen lead measurements. It will be appreciated that other lead configurations are also possible and are similarly applicable to the embodiments described herein.

Each lead measurement is an ECG signal that undergoes a corresponding ST segment measurement with a ST segment value associated therewith. Furthermore, each lead measurement is mapped to a different corresponding region (i.e., anatomical area) of the heart for the monitoring of that region. As a result, each monitored region of the heart has a corresponding ECG signal whose ST segment can be continuously yet separately monitored for normal, depressed, and elevated ST segment conditions. For a 12-lead ECG configuration, twelve different regions of the heart are monitored and each heart region has its own ST segment measurement indicative of a normal, depressed, or elevated ST segment in that region.

Normal, depressed, and/or elevated ST segments, which may be present in any combination among the monitored heart regions, may be indicative of a physiological condition of the patient, with different combinations indicating possibly different physiological conditions, such as infarction or ischemia. Depending on the degree of the ischemic or infarction event, the ECG will show an ST depression (negative voltage) or an ST elevation (positive voltage). For the left anterior descending coronary artery, this typically appears as ST elevation maximal in precordial leads V2 or V3, and for the right coronary artery, as ST elevation maximal in limb leads aVF or III. However, except when the left circumflex coronary artery is dominant (supplies the posterior descending artery), its acute occlusion is represented instead by ST depression maximal in leads V2 or V3. The elevation or depression of the ST segment in a particular lead combination will point to the particular area of the heart that experiences an ischemic event.

The patient monitor 7 is configured to display a rotatable 3D anatomical representation of the heart in which each heart region that corresponds to a lead measurement is visually identified in the 3D anatomical representation such that it is visually distinct from other heart regions. In addition, each heart region identified in the 3D anatomical representation has a visual marker or visual indicator that has a characteristic unique to a measured value of its the ST segment. The measured value of the ST segment may be, for example, an amplitude of the ST segment, which can be correlated to a normal, depressed, or elevated ST segment and to a degree of normality, depression, or elevation within predefined ranges corresponding thereto. For example, a visual indicator may be a color, a color intensity, a color shading, a region outline, a region shape, or a combination thereof such that a measured amplitude of an ST segment is visually represented and readily discernable in each heart region. As a result, a region of the heart in which an abnormal ST segment event (e.g., a depression or an elevation) occurs can be easily identified, as well as regions in which normal ST segment events are present.

For carrying out an ECG test, a variable number of ECG electrodes are positioned on a patient in a way that the electrodes form a predefined arrangement, e.g., accordingly to “Einthoven”, “Goldberger” and “Wilson”, EASI, “Frank” or others. A 12-lead ECG may have, for example, six vertical leads of a 12-lead-ECG, namely aVF, III, aVL, I, aVR and II (listed according to a clockwise arrangement on a patient's body). Thereby the bipolar “Einthoven” leads I, II and III and the unipolar “Goldberger” leads aVR, aVL and aVF are used. The displayed values for each lead can be obtained from a mathematical linear combination of the values of the electrical voltages obtained from the ECG electrodes.

Likewise, six axes relating to six horizontal “Wilson” leads of a 12-lead-ECG, namely V1, V2, V3, V4, V5, and V6 are used (listed according to a horizontal arrangement across a patient's body from left to right). Again, the position of these axes and their angles represent the location of its corresponding ECG electrode on the patient's body during the ECG test. Not all the leads need to be measured, as some of the leads can be derived from a linear combination of other leads. For example, in a 7-lead ECG configuration, aVF, III, aVL, I, aVR and Il leads may be present and one of the V1, V2, V3, V4, V5, and V6 leads may be present depending on the placement of a corresponding ECG electrode on the patient's body. For example, if an ECG electrode is placed at a position corresponding to lead V2, then lead V2 will be measured. Likewise, if an ECG electrode is placed at a position corresponding to lead V4, then lead V4 will be measured. Additional lead measurements can be added by adding additional electrodes to the patient's body and selecting the desired ECG configuration at the patient monitor 7 where the selected ECG configuration corresponds to an ECG algorithm used by the patient monitor 7 to derive the appropriate lead measurements.

The position of each heart region displayed on the rotatable 3D anatomical representation is configured based on the number of electrodes used and their location on the patient's body (i.e., by the selected ECG configuration).

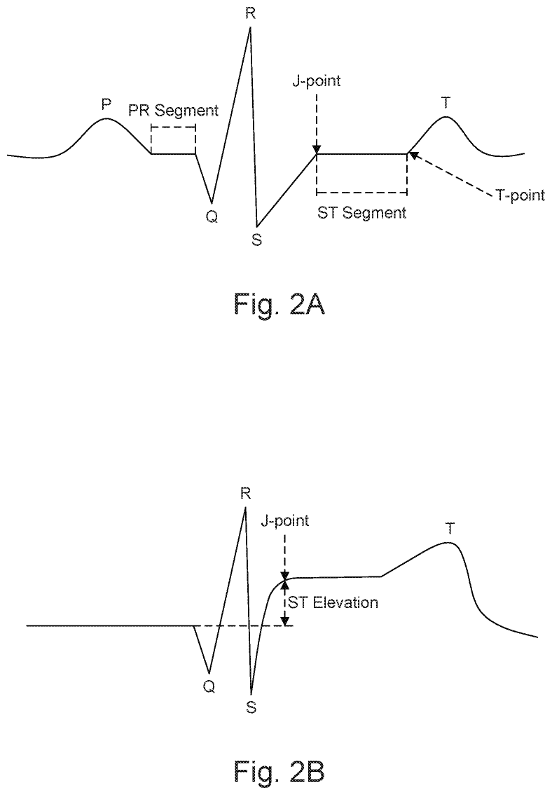

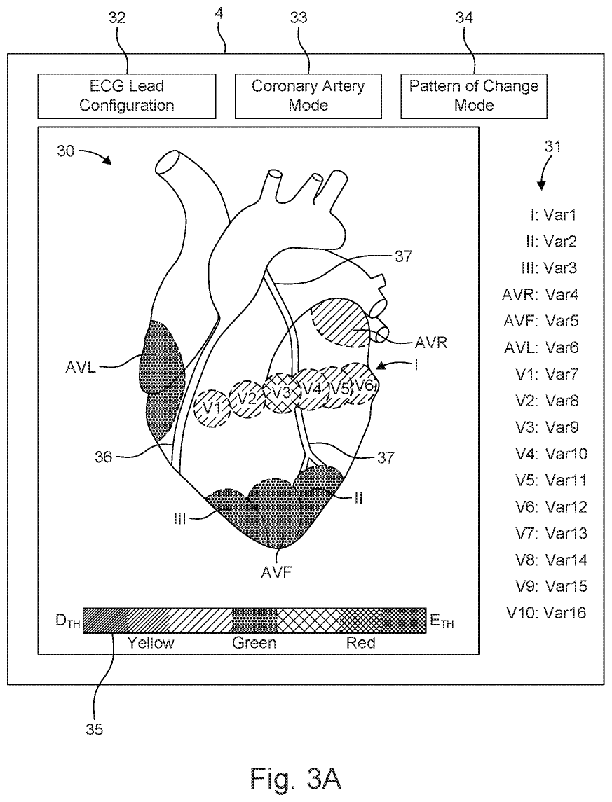

FIG. 2A is a plot of an example cardiac cycle waveform of an ECG signal showing the Q, R, S, and T waves, the J-point, and the ST segment. Each cardiac cycle waveform can have a QRS complex and an R-peak, S-peak, T-peak, J-point, and an ST segment. The QRS complex characterizes the depolarization of the right and left ventricles of the human heart. The ST segment characterizes an interval between ventricular depolarization and repolarization and occurs after the QRS complex. The ST segment corresponds to a line on an electrocardiogram that begins with the end of the QRS complex and ends at the beginning of the T wave (i.e., repolarization). The height of the ST segment is normally equal to that of the PR segment and/or the TP segment (sometimes referred to herein as a baseline relative to which deviation of the ST segment is measured).

For example, the patient monitor 7 via processor 3 may be configured to monitor the cardiac cycle waveform for each measurement lead (i.e., for each monitored heart region) and use the J-point of each measurement lead as the amplitude of the corresponding ST segment and/or to use the J-point to calculate the ST segment deviation (e.g., elevation or depression in relation to the baseline). Thus, the patient monitor 7 detect and measure the baseline, detect and measure the J-point, and calculate the ST segment amplitude or baseline deviation from the two measurements. ST segment elevation can be found in patients with acute myocardial infarction and other conditions while ST segment depression is an indicator of coronary ischemia.

The TP segment of a cardiac cycle waveform is the segment defined by the end of the T wave and start of a next P wave in the cardiac cycle waveform. The PR segment of a cardiac cycle waveform is the segment defined by an end of a P wave and start of a QRS complex in the cardiac cycle waveform.

FIG. 2B is a plot of an example cardiac cycle waveform of an ECG signal showing a ST elevation. In this case, the J-point is elevated above the baseline.

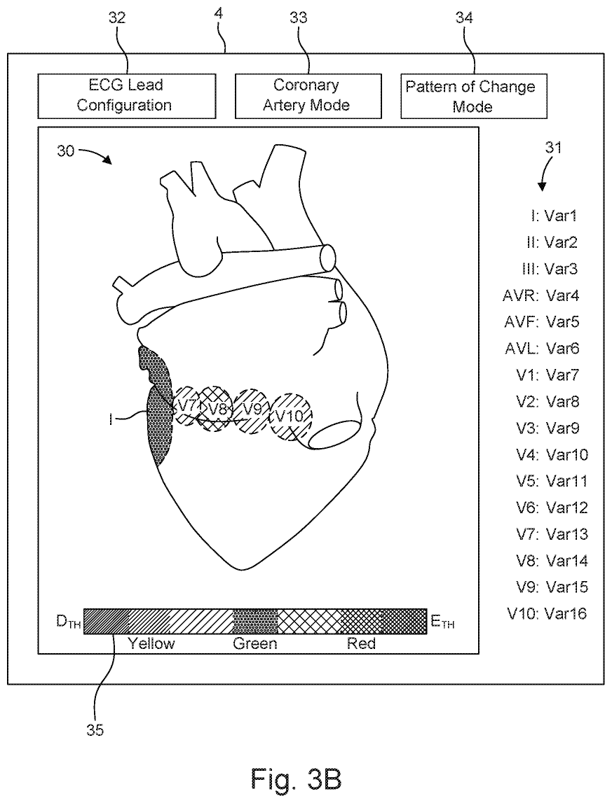

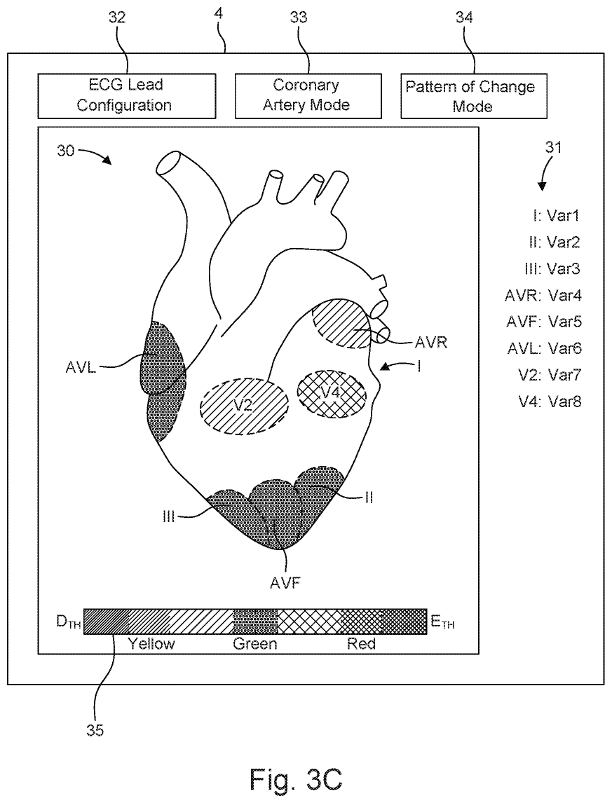

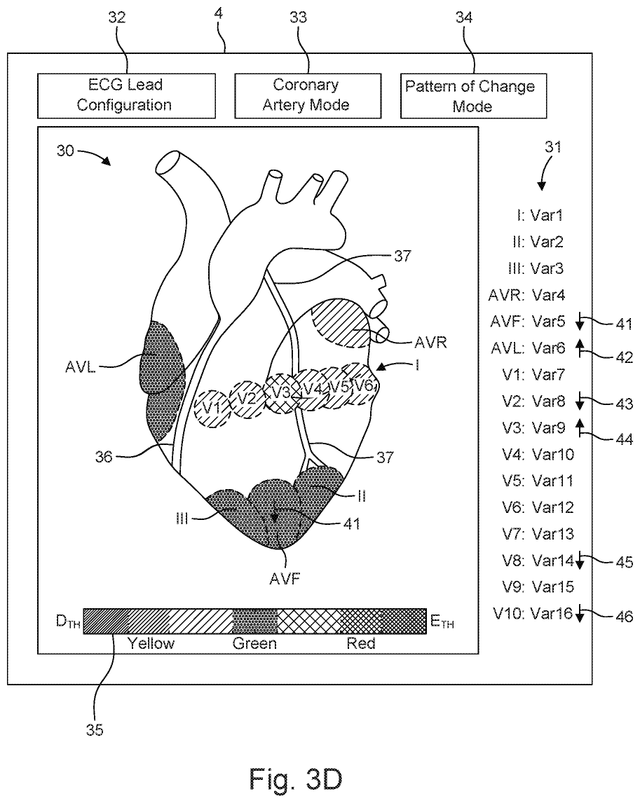

FIGS. 3A and 3B illustrate a display 4 of a patient monitor 7 configured to display a rotatable 3D anatomical representation of a heart 30 including visually identifiable regions each corresponding to a different ECG lead measurement according to one or more embodiments. In particular, FIG. 3A shows a frontal view of the 3D anatomical representation of the heart 30 that is adapted according to a 12-lead or 16-lead ECG configuration. In this view, the 3D anatomical representation of the heart 30 includes eleven visible heart regions II, III, AVF, AVL, AVR, V1, V2, V3, V4, V5, and V6 each corresponding to a different ECG lead measurement that a processor (e.g., processor 3) derives from the ECG electrodes 17 that are attached to a patient's body. Each heart region is delineated by a boundary that makes it discernable from the other heart regions. It is noted that heart region I corresponding to lead I is visible from a side view or posterior view of the heart 30.

FIG. 3B is a posterior view of the 3D anatomical representation of the heart 30 adapted to a 16-lead ECG configuration with heart regions I, V7, V8, V9, and V10 being visible on the display 4. Heart region I is also present in a 12-lead ECG configuration, with heart regions V7, V8, V9, and V10 being generated in or superimposed onto the 3D anatomical representation of the heart 30 in the 16-lead ECG configuration by the addition of additional ECG electrodes. Again, each heart region visually marked on the 3D anatomical representation of the heart 30 corresponds to a different ECG lead measurement that a processor (e.g., processor 3) derives from the ECG electrodes 17 that are attached to a patient's body.

FIG. 3C is a frontal view of the 3D anatomical representation of the heart 30 adapted to an 8-lead ECG configuration with heart regions AVF, AVL, AVR, II, III, V2 and V4 being visible on the display 4.

The display 4 is also configured to display real-time numerical values 31 of ST segment measurements acquired for each of the ECG leads (i.e., for each of the heart regions). This enables the numerical values to be observed for all leads regardless of the orientation of the 3D anatomical representation of a heart 30. The numerical values, also referred to as variables, are rational numbers that may be negative to indicate an ST depression, neutral to indicate a normal (e.g., substantially flat ST segment), or positive to indicate an ST elevation. Each numerical value is a variable that represents the amplitude of its ST segment measurement, which may be a deviation amount of its J-point from its baseline value. Some leads may be more depressed or more elevated than others. Thus, each numerical value has a magnitude that corresponds to how strongly its ST segment deviates from its baseline, which corresponds to how critical an abnormal ST segment event is should one be present. In the case of a 12-lead ECG configuration, twelve variables Var1-Var12 are displayed proximate to their respective lead symbol. In the 16-lead ECG configuration, sixteen variables Var1-Var16 are displayed proximate to their respective lead symbol.

The 3D anatomical representation of the heart 30 is rotatable by 360 degrees in any direction so that the heart can be viewed from any vantage point. A user may rotate the 3D anatomical representation of the heart 30 by manipulating a means for user input, such as the touch screen of the display 4 or operating a mouse, a keyboard, etc. A user may also manipulate one or more GUI icons 32-34 to configure the 3D anatomical representation of the heart 30 according to a desired ECG lead configuration or set one or more display modes.

For example, GUI icon 32 may be used by a user to select from multiple ECG lead configurations, which in turn causes the processor 3 to generate the 3D anatomical representation of the heart 30 to have the appropriate marked heart regions.

GUI icon 33 may be used by a user to enable and disable a coronary artery mode in which the coronary arteries are anatomically superimposed onto the 3D anatomical representation of the heart 30 that may be used to help a user locate where they are looking on the heart and if there is an infarct or ischemia. In FIG. 3A, the right coronary artery 36 and the left coronary artery 37 are superimposed onto the heart 30 in response to the coronary artery mode being enabled. In this mode, depending upon the ST value associated with a particular lead, the vessels around that area will be visually highlighted to be impacted or not. As an example, if the ST values for lead V3 and/or lead V4 is depressed below the baseline, it would indicate that the left coronary artery 37 is blocked and the blood flow to that region proximate to leads V3 and V4 is interrupted. In this case, the area of the affected coronary artery that is affected/diseased may be visually highlighted by a color change or other visual indicator. The processor 3 may determine which coronary artery is affected and which portion of that coronary artery is affected according to the ST values of the leads.

GUI icon 34 may be used by a user to enable and disable a progression mode which further adds the capability of tracking a pattern of changes in the ST segments over time. For example, additional icons or overlays may be generated on the display 4 to indicate a pattern of change in the ST segment for each lead.

A visual indicator scale 35 is also generated on the display 4 to act as a key for interpretating visual indicators generated at each heart region. A visual indicator is superimposed onto each heart region of the 3D anatomical representation of the heart 30 and is visible when the heart region is in view. The appearance of each visual indicator is correlated with a corresponding numerical value among variables Var1-Var16 in relation to the visual indicator scale 35 that progresses from a lower limit threshold DTH (i.e., a depression lower limit) to an upper limit threshold ETH (i.e., an elevation upper limit). The appearance of each visual indicator changes in real time as an ST segment measurement for a corresponding lead changes.

In one example, the visual indicator scale 35 is a color scale and a visual indicator is a colored area overlaid on each heart region. A normal status (i.e., a normal ST segment) may be assigned one color (e.g., green), a depressed ST segment may be assigned another color (e.g., yellow), and an elevated ST segment may be assigned another color (e.g., red), the colors of which are indicated on the visual indicator scale 35. If an ST segment of a lead is measured as normal, its corresponding heart region is illuminated green. On the other hand, if an ST segment of a lead is measured as abnormal, its corresponding heart region is illuminated as yellow or red depending on whether the abnormality is a depressed ST segment or an elevated ST segment.

Additionally, the intensity of a colored area may change based on the magnitude of the ST segment deviation from the baseline. For example, the intensity of the yellow color may gradually increase on a sliding scale as the measured variable moves away from the normal region towards the lower limit threshold DTH and the intensity of the red color may gradually increase on a sliding scale as the measured variable moves away from the normal region towards the upper limit threshold ETH. Each heart region has its own color and color intensity relative to its ST segment measurement. As an ST segment measurement changes over time, so does the color and/or color intensity of the corresponding heart region. As a result, the visible heart regions as a whole may be illuminated with different colors and color intensities at any given moment in order to provide a real-time status of a patient's condition as the ST segment measurements are performed for each lead.

The values assigned to the normal ST segment range may be defined by demographics data of the patient (e.g., age, height, weight) and are represented by a specific middle range color. Likewise, the value for the lower limit threshold DTH and the value for the upper limit threshold ETH may also be defined by the demographics data of the patient. In this way, the regions of the color scale and the progression of color intensities can be adapted to a specific patient with the color scale representing a range from a lower limit to an upper limit threshold that is focused on certain range of patient. The same color scale is applicable to all leads.

Should the lower limit threshold DTH or the upper limit threshold ETH be reached or exceeded at any lead, the patient monitor 7 may generate additional alarms or visual triggers. For example, the patient monitor 7 may cause a corresponding colored area to flash or blink. It may also trigger an audible alarm.

It will be further appreciated that a different color scale may be used to represent normal and abnormal ST segment conditions or that a different visual indicator may be used altogether or in combination therewith. For example, each heart region may be assigned a shape that is adjusted based on the ST segment measurement or a line type that encloses each heart region may change based on the ST segment measurement. Accordingly, the visually distinct regions displayed on the 3D anatomical representation of the heart 30 and associated with the plurality of ECG signals and change in appearance based on their respective real-time ST segment measurements, and particularly, based on whether their respective real-time ST segment measurement is depressed, normal, or elevated.

The 3D anatomical representation of the heart 30 and the visual indicator scale 35 enable users of different levels of clinical knowledge to quickly and clearly identify where the focus points on the heart are relative to an abnormal condition, diagnose a possible physiological condition of the patient based on the status of each color area (i.e., based on the appearance of each visually distinct region), and direct any treatment accordingly.

FIG. 3D illustrates a display 4 of a patient monitor 7 with a pattern of change mode enabled via the GUI icon 34. When the pattern of change mode is enabled, pattern of change indicators (e.g., icons) are displayed either next to their respective measured numerical values 31 or may be overlayed over their respective heart region within the 3D anatomical representation of the heart 30. The pattern of change indicators indicate how an ST segment measurement in a respective heart region has changed over time. For example, a pattern of change indicator may indicate that an amplitude of an ST segment measurement has increased or decreased over time.

In one example, the processor 3 may be configured to periodically capture snapshots of the ST segment measurements (i.e., of the numerical values of variables Var1-Var16) and compare the most recent captured values to captures values from a previous snapshot or to baseline values of variables Var1-Var16. The snapshots may be triggered by the processor 3 either automatically at preset time intervals or may be triggered manually by user input. A baseline value of a variable may be one captured at the start of patient monitoring (e.g., before clinical intervention or treatment). This may be particularly useful when comparing ST segment measurements taken after clinical intervention or treatment to baseline values set prior to clinical intervention or treatment to assess how the patient is responding to the clinical intervention or treatment. It is also possible to set a new baseline after clinical intervention or treatment has been performed in order to further assess how the patient is responding to the clinical intervention or treatment.

During a pattern change analysis, the processor 3 is configured to compare each variable taken at a most recent snapshot with a previous value of its variable or the variable's baseline value. For the comparison, the processor 3 may determine a comparison result by determining whether a current value of a variable is less than, equal to, or greater than a previous value of its variable or the variable's baseline value. If the processor 3 determines that the comparison result is a “less than” result, it may control the display 4 to display a decreasing pattern of change indicator (e.g., a downward arrow) on the display 4 next to the variable or overlaid on the corresponding heart region. If the processor 3 determines that the comparison result is an “equal to” or neutral result, it may control the display 4 to display a neutral pattern of change indicator (e.g., a flat line) on the display 4 next to the variable or overlaid on the corresponding heart region or have no pattern of change indicator icon displayed for that variable. If the processor 3 determines that the comparison result is a “greater than” result, it may control the display 4 to display an increasing pattern of change indicator (e.g., an upward arrow) on the display 4 next to the variable or overlaid on the corresponding heart region.

Alternatively, for the comparison, the processor 3 may determine a differential value (i.e., a delta value) representative of a difference between a current value a previous value of its variable or between the variable's baseline value. The processor 3 may then compare each differential value to an upper (positive) threshold value and a lower (negative) threshold value. If a differential value is equal to or less than the lower threshold value, the processor 3 may control the display 4 to display the decreasing pattern of change indicator. If a differential value is equal to or greater than the upper threshold value, the processor 3 may control the display 4 to display the increasing pattern of change indicator. If a differential value is between the lower and upper threshold values, the processor 3 may control the display 4 to display the neutral pattern of change indicator or no pattern of change indicator icon. Thus, values between the lower and upper threshold values define a predetermined margin of no significant change.

In FIG. 3D, pattern of change indicators 41-46 are shown, indicating that there has been a pattern change at leads AFV, AVL, V2, V3, V8, and V10, whereas no change or no change outside the predetermined margin has occurred in the remaining leads. As an example, the pattern of change indicator 41 indicates that a patient's ST segment for lead AVF has decreased over time. If heart region AVF was previously red, this may indicate an improvement in this region since the region is now green. The pattern of change indicator 42 indicates that a patient's ST segment for lead AVL has increased over time. If heart region AVL was previously yellow, this may indicate an improvement in this region since the region is now green. Thus, each of the pattern of change indicators 41-46 may indicate the direction in which the ST segment measurements are progressing over time.

Alternatively, the pattern of change indicators 41-46 may indicate whether a condition has improved (i.e., a variable has moved closer to neutral) in a respective heart region/lead, whether a condition has become worse (i.e., a variable has moved further away from neutral), or whether a condition has remained the same or substantially the same since a previous snapshot or relative to the baseline values of variables Var1-V16. For example, an upward arrow may indicate that a condition has improved and a downward arrow may indicate that a condition has gotten worse over time. A neutral icon or no icon may indicate no change in condition for a respective heart region/lead.

In view of the above, processor 3 or an external processor (e.g., processor 12) is configured to analyze ST segment measurements for each lead/heart region and control the display 4 to adapt the rotatable 3D anatomical representation of a heart 30, the numerical values 31, and the pattern of change indicators 41-46 in real time in accordance with continuous ST segment measurements. Moreover, the rotatable 3D anatomical representation of a heart 30 is adapted with changing visual indictors for each heart region being targeted for measurement as the ST segment measurements change over time. Each heart region is visually identifiable and the status of each heart region is readily apparent to a user due to their respective visual indicator. A user may focus on different anatomical views of the heart by rotating the rotatable 3D anatomical representation of a heart 30 in any direction. Additionally, other physiological parameters, such as cardiac output and heart rate, may also be simultaneously displayed on display 4 to provide a user with addition patient information pertinent to diagnosis and treatment.

While various embodiments have been disclosed, it will be apparent to those skilled in the art that various changes and modifications can be made which will achieve some of the advantages of the concepts disclosed herein without departing from the spirit and scope of the present disclosure. It will be obvious to those reasonably skilled in the art that other components performing the same functions may be suitably substituted. It is to be understood that other embodiments may be utilized and structural or logical changes may be made without departing from the scope of the present disclosure. It should be mentioned that features explained with reference to a specific figure may be combined with features of other figures, even in those not explicitly mentioned. Such modifications to the general inventive concept are intended to be covered by the appended claims and their legal equivalents.

Furthermore, the following claims are hereby incorporated into the detailed description, where each claim may stand on its own as a separate example embodiment. While each claim may stand on its own as a separate example embodiment, it is to be noted that-although a dependent claim may refer in the claims to a specific combination with one or more other claims -other example embodiments may also include a combination of the dependent claim with the subject matter of each other dependent or independent claim. Such combinations are proposed herein unless it is stated that a specific combination is not intended. Furthermore, it is intended to include also features of a claim to any other independent claim even if this claim is not directly made dependent to the independent claim.

It is further to be noted that methods disclosed in the specification or in the claims may be implemented by a device having means for performing each of the respective acts of these methods. For example, the techniques described in this disclosure may be implemented, at least in part, in hardware, software, firmware, or any combination thereof. For example, various aspects of the described techniques may be implemented within one or more processors, including one or more microprocessors, DSPs, ASICs, or any other equivalent integrated or discrete logic circuitry, as well as any combinations of such components.

Further, it is to be understood that the disclosure of multiple acts or functions disclosed in the specification or in the claims may not be construed as to be within the specific order. Therefore, the disclosure of multiple acts or functions will not limit these to a particular order unless such acts or functions are not interchangeable for technical reasons. Furthermore, in some embodiments a single act may include or may be broken into multiple sub acts. Such sub acts may be included and part of the disclosure of this single act unless explicitly excluded.

1. A physiological monitoring device, comprising:
a sensor interface configured to receive sensor signals from a plurality of electrocardiogram (ECG) sensors connected to a patient;
a display configured to display information related to the patient; and
at least one processor configured to receive the sensor signals and derive a plurality of ECG signals therefrom, wherein each ECG signal is a cardiac cycle waveform associated with a different region of a heart, wherein the at least one processor is further configured to measure an ST segment of each ECG signal in real time to acquire a plurality of real-time ST segment measurements and control the displayed information based on the plurality of real-time ST segment measurements,
wherein the displayed information includes a rotatable 3D anatomical representation of the heart that includes a plurality of visually distinct regions that are associated with the plurality of ECG signals, wherein each visually distinct region is associated with a different ECG signal of the plurality of ECG signals,
wherein each visually distinct region comprises at least one visual characteristic that is configured to change based on a real-time ST segment measurement of the ECG signal associated therewith,
wherein each measured ST segment of each ECG signal is identifiable as a depressed ST segment, a normal ST segment, or an elevated ST segment according to the at least one visual characteristic of a corresponding visually distinct region of the plurality of visually distinct regions, and
wherein the at least one processor is configured to adapt the at least one visual characteristic of each visually distinct region in real-time based on the real-time ST segment measurement associated therewith to visually indicate that the real-time ST segment measurement associated therewith is depressed, normal, or elevated.

2. The physiological monitoring device of claim 1, wherein the at least one visual characteristic includes a color overlay that overlaps its corresponding visually distinct region, wherein the at least one processor is configured to adapt a color of each color overlay to visually indicate that the real-time ST segment measurement associated therewith is depressed, normal, or elevated.

3. The physiological monitoring device of claim 2, wherein the at least one processor is configured to associate a depressed ST segment with a first color, a normal ST segment with a second color, and an elevated ST segment with a third color.

4. The physiological monitoring device of claim 2, wherein the at least one processor is configured to adapt a color intensity of each color overlay to visually indicate a magnitude of the real-time ST segment measurement associated therewith.

5. The physiological monitoring device of claim 2, wherein the displayed information includes a color scale that indicates a color progression from an ST depression lower limit to an ST elevation upper limit.

6. The physiological monitoring device of claim 5, wherein the at least one processor is configured to set the ST depression lower limit and the ST elevation upper limit based on demographic information of the patient.

7. The physiological monitoring device of claim 2, wherein the displayed information includes a visual indicator scale that indicates a visual indicator progression from an ST depression lower limit to an ST elevation upper limit.

8. The physiological monitoring device of claim 1, wherein the displayed information includes a numeral value for each visually distinct region that represents the real-time ST segment measurement associated therewith.

9. The physiological monitoring device of claim 1, wherein each ECG signal corresponds to a different lead of a plurality of leads of an ECG lead configuration, and each visually distinct region displayed on the rotatable 3D anatomical representation of the heart is uniquely associated with one of the plurality of leads.

10. The physiological monitoring device of claim 9, wherein the at least one processor is configured to determine the ECG lead configuration and enable a set of visually distinct regions on the rotatable 3D anatomical representation of the heart based on the ECG lead configuration.

11. The physiological monitoring device of claim 1, wherein the plurality of visually distinct regions are rotatable into and out of view based on an orientation of the rotatable 3D anatomical representation of the heart.

12. The physiological monitoring device of claim 1, wherein the displayed information includes coronary artery overlays that are superimposed onto the rotatable 3D anatomical representation of the heart, wherein the at least one processor is configured to selectively enable and disable the coronary artery overlays in response to user input.

13. The physiological monitoring device of claim 1, wherein the displayed information includes pattern of change indicators, each associated with a different ECG signal of the plurality of ECG signals and indicative of a change over time in a real-time ST segment measurement of the ECG signal associated therewith.

14. The physiological monitoring device of claim 13, wherein each pattern of change indicator indicates whether the real-time ST segment measurement of the ECG signal associated therewith has improved, remains substantially the same, or worsened with respect to a previous ST segment measurement of the ECG signal associated therewith or with respect to a baseline value.

15. The physiological monitoring device of claim 13, wherein the at least one processor is configured to periodically capture the plurality of real-time ST segment measurements in memory, compare the real-time ST segment measurements with corresponding previous real-time ST segment measurements, and generate the pattern of change indicators based on the comparison results.

16. An electrocardiogram (ECG) system, comprising:
a display configured to display information related to the patient;
at least one processor configured to receive physiological signals collected from a plurality of electrocardiogram (ECG) sensors connected to a patient and derive a plurality of ECG signals therefrom, wherein each ECG signal is a cardiac cycle waveform associated with a different region of a heart, wherein the at least one processor is further configured to measure an ST segment of each ECG signal in real time to acquire a plurality of real-time ST segment measurements and control the displayed information based on the plurality of real-time ST segment measurements,
wherein the displayed information includes a rotatable 3D anatomical representation of the heart that includes a plurality of visually distinct regions that are associated with the plurality of ECG signals, wherein each visually distinct region is associated with a different ECG signal of the plurality of ECG signals,
wherein each visually distinct region comprises at least one visual characteristic that is configured to change based on a real-time ST segment measurement of the ECG signal associated therewith,
wherein each measured ST segment of each ECG signal is identifiable as a depressed ST segment, a normal ST segment, or an elevated ST segment according to the at least one visual characteristic of a corresponding visually distinct region of the plurality of visually distinct regions, and
wherein the at least one processor is configured to adapt the at least one visual characteristic of each visually distinct region in real-time based on the real-time ST segment measurement associated therewith to visually indicate that the real-time ST segment measurement associated therewith is depressed, normal, or elevated.

17. The ECG system of claim 16, wherein the at least one visual characteristic includes a color overlay that overlaps its corresponding visually distinct region, wherein the at least one processor is configured to adapt a color of each color overlay to visually indicate that the real-time ST segment measurement associated therewith is depressed, normal, or elevated.

18. The ECG system of claim 17, wherein the at least one processor is configured to associate a depressed ST segment with a first color, a normal ST segment with a second color, and an elevated ST segment with a third color.

19. The ECG system of claim 17, wherein the at least one processor is configured to adapt a color intensity of each color overlay to visually indicate a magnitude of the real-time ST segment measurement associated therewith.

20. The ECG system of claim 16, further comprising:
a physiological monitoring device comprising a sensor interface configured to receive the physiological signals, the display, and a first processor of the at least one processor; and
a network device communicatively coupled to the physiological monitoring device and comprising a second processor of the at least one processor,
wherein the first processor and the second processor share a processing load for at least one of acquiring the plurality of real-time ST segment measurements and controlling the displayed information based on the plurality of real-time ST segment measurements.

21.-25. (canceled)
